(19) United States
(12) Patent Application Publication
(10) Pub. No.: US 2024/0074797 A1
(43) Pub. Date: Mar. 7, 2024

(54) Capped Distraction/Compression Rod System

(71) Applicant: Matthew Amarante, Chatham, NJ (US)

(72) Inventor: Matthew Amarante, Chatham, NJ (US)

(21) Appl. No.: 18/071,600

(22) Filed: Nov. 29, 2022

Related U.S. Application Data
(60) Provisional application No. 63/285,074, filed on Dec. 1, 2021.

(51) Int. Cl. A61B 17/70

(52) U.S. Cl. CPC A61B 17/7079; A61B 2017/0256

(57) ABSTRACT

For spinal surgery, an improved rod system is designed to efficiently provide defined amounts of compression/distraction forces without the need for additional tools or steps to manipulate the screws previously implanted into a patient's vertebrae. The improved rod system may engage with two or more screws and provide combinations of distraction, compression, and no force to the different screws, by varying the sizes of the rod system and its inner segments.

### BACKGROUND AND FIELD OF THE INVENTION

The present disclosure pertains generally to medical tools used in spinal surgery.

In the field of spinal surgery, various treatments and procedures use screws that are placed into the pedicles of a patient's spine or other parts of the vertebrae. In minimally invasive approaches, small incisions are used to provide the surgeon access to the spine for placing screws. Screw extenders and reinforcing sleeves are connected to the screws to assist with various minimally invasive procedures. Rods are then inserted to engage with and fix to the screws.

Conventionally, compression and distraction forces may also be applied to the spine using various instruments and tools to manipulate the screws. These tools include fulcrum devices, pliers, rack compressor/distractor devices, and compressor arms, which engage with the screw components to provide compression and distraction forces on the patient's spine.

These conventional tools and procedures can be complicated, time-consuming, and ineffective, involving a number of interconnected tools and specialized parts that require careful placement, use, and monitoring. What is needed is a more efficient system and method for applying compression and distraction forces as part of minimally invasive spinal procedures. Furthermore, this improved system can be adapted to any spinal hardware manufacturer and applied to traditional open surgical approaches to the spine.

### SUMMARY OF THE INVENTION

Embodiments of the present disclosure provide improved rods that are designed to provide compression or distraction forces as part of placement. One benefit of the embodiments described herein is that the number of tools required for treatment and various procedures are reduced. Another benefit of the embodiments described herein is that the number of steps required for treatment and various procedures are reduced or simplified. Another benefit of the embodiments described herein is that conventional benefits from minimally invasive spinal procedures are maintained and the embodiments can be deployed with and adapted to any number of conventional surgery settings without requiring new tool sets or training to use.

According to one embodiment of the present disclosure, an improved rod includes inner end caps that separate the rod length into three segments, two of which engage respectively with two screws placed in the pedicles of a patient's spine.

According to another embodiment of the present disclosure, an improved rod includes inner end caps that separate the rod length into five segments, three of which engage respectively with three screws placed in the pedicles of a patient's spine.

According to another embodiment of the present disclosure, an improved rod is placed to engage with two screw extenders and sized according to measurements of a patient's spine to provide a defined amount of distraction or compression between two screws.

According to another embodiment of the present disclosure, an improved rod is placed to engage with three screw extenders and sized according to measurements of a patient's spine to provide a defined amount of distraction or compression between three screws.

According to another embodiment of the present disclosure, an improved rod is placed to engage with three screw extenders and sized according to measurements of a patient's spine to provide two different defined amounts of distraction or compression between three screws.

According to another embodiment of the present disclosure, an improved rod is placed to engage with three screw extenders and sized according to measurements of a patient's spine to provide a combination of compression/distraction/no force amongst the three screws.

Additional features and advantages will be made apparent from the following detailed description of the embodiments, as illustrated and described below.

### BRIEF DESCRIPTION

The foregoing and other aspects of the embodiments of the present disclosure are best understood from the following detailed description when read in connection with the accompanying drawings. For purposes of illustration and description, embodiments of the present disclosure have been illustrated, but the invention is not limited to the specific instrumentalities as illustrated and described, as would be understood by those skilled in the art.

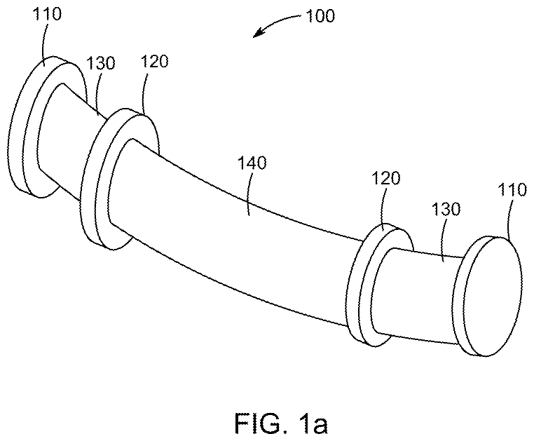

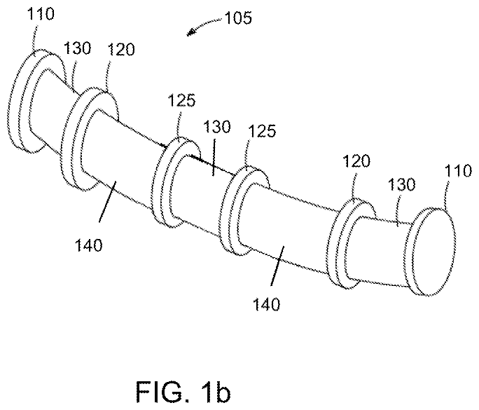

FIG. 1a depicts an embodiment of the capped distraction/compression rod with two screw head rod segments.

FIG. 1b depicts an embodiment of the capped distraction/compression rod with three screw head rod segments.

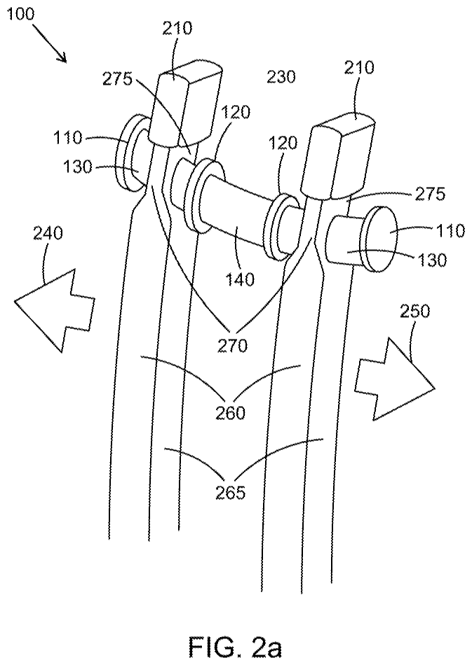

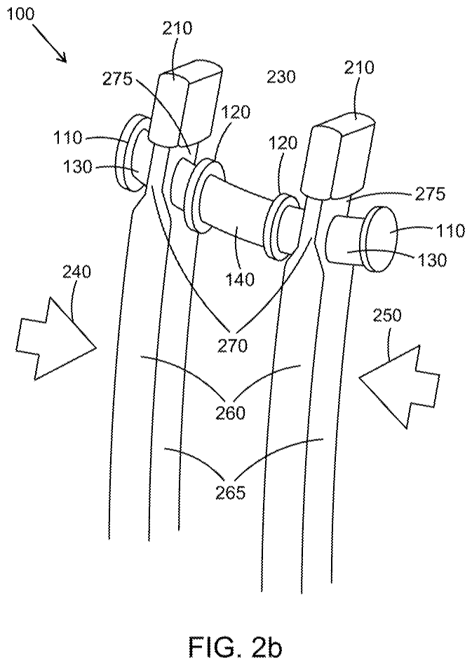

FIG. 2a depicts an embodiment of the capped distraction/compression rod engaging with two sets of screw tab extenders near a set of two extender caps and imparting a distraction force.

FIG. 2b depicts an embodiment of the capped distraction/compression rod engaging with two sets of screw tab extenders near a set of two extender caps and imparting a compression force.

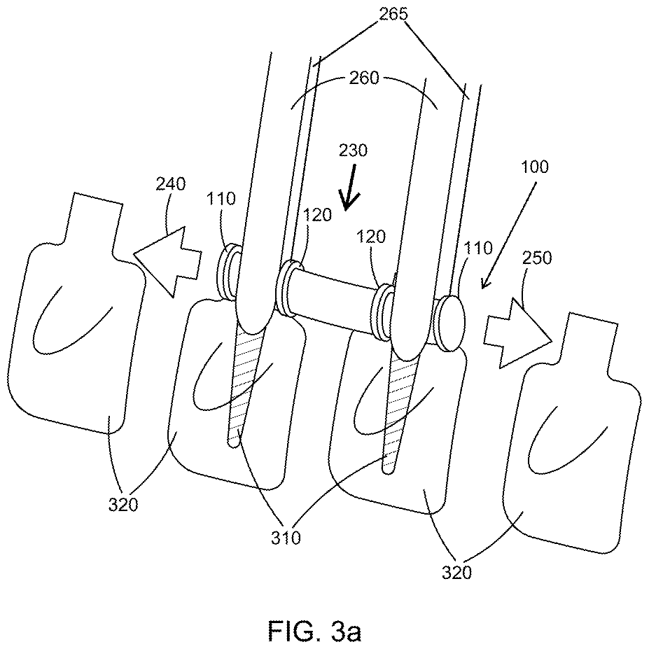

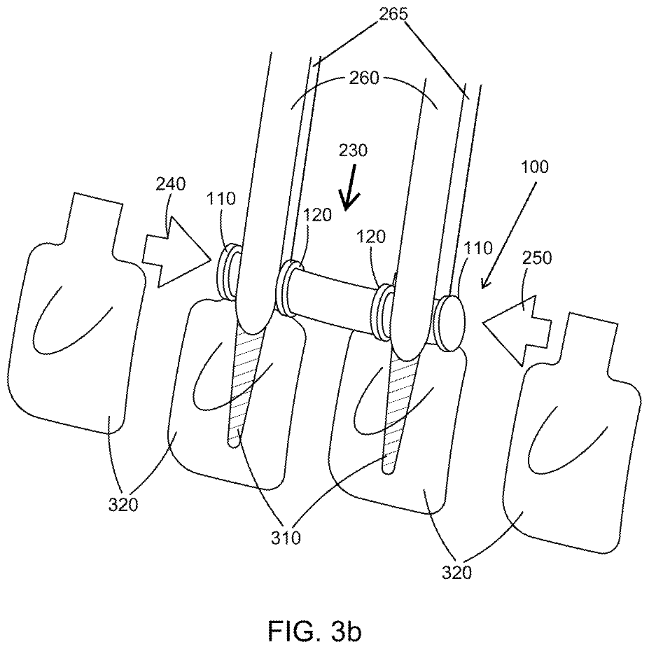

FIG. 3a depicts an embodiment of the capped distraction/compression rod engaging with a set of two multi-axial screws and imparting a distraction force.

FIG. 3b depicts an embodiment of the capped distraction/compression rod engaging with a set of two multi-axial screws and imparting a compression force.

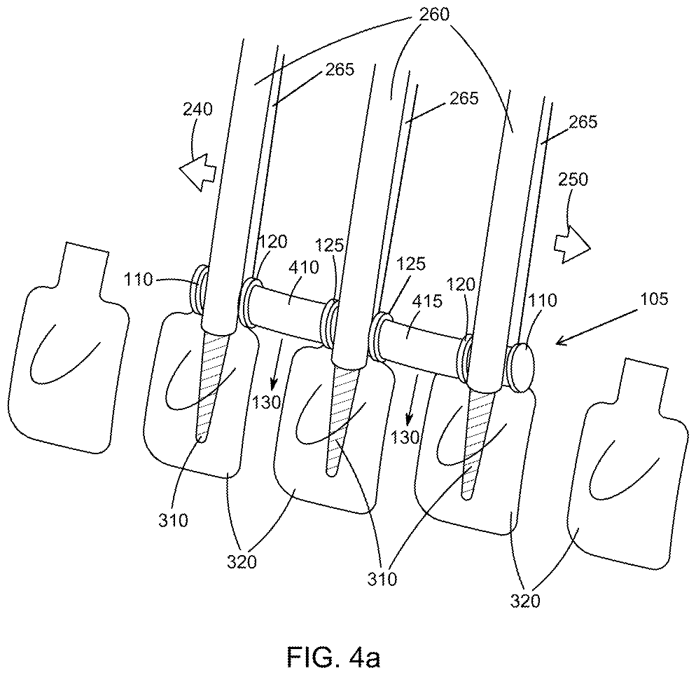

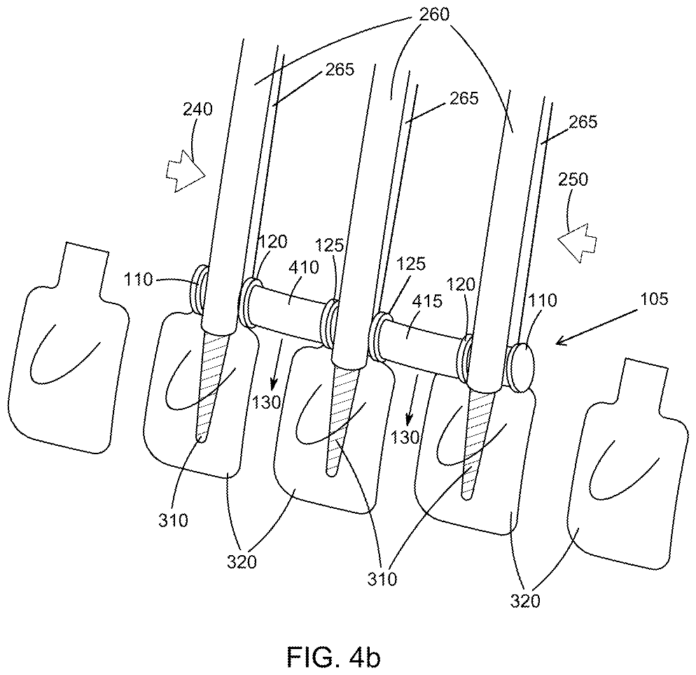

FIG. 4a depicts an embodiment of the capped distraction/compression rod engaging with a set of three multi-axial screws and imparting a distraction force.

FIG. 4b depicts an embodiment of the capped distraction/compression rod engaging with a set of three multi-axial screws and imparting a compression force.

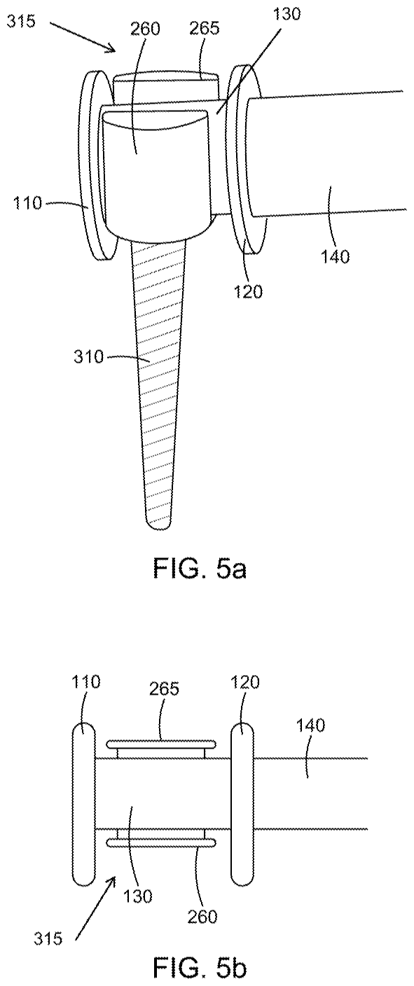

FIG. 5a depicts a side profile view of one end of a capped distraction/compression rod seated into the screw head of a multi-axial screw.

FIG. 5b depicts a top profile view of one end of a capped distraction/compression rod seated into the screw head of a multi-axial screw.

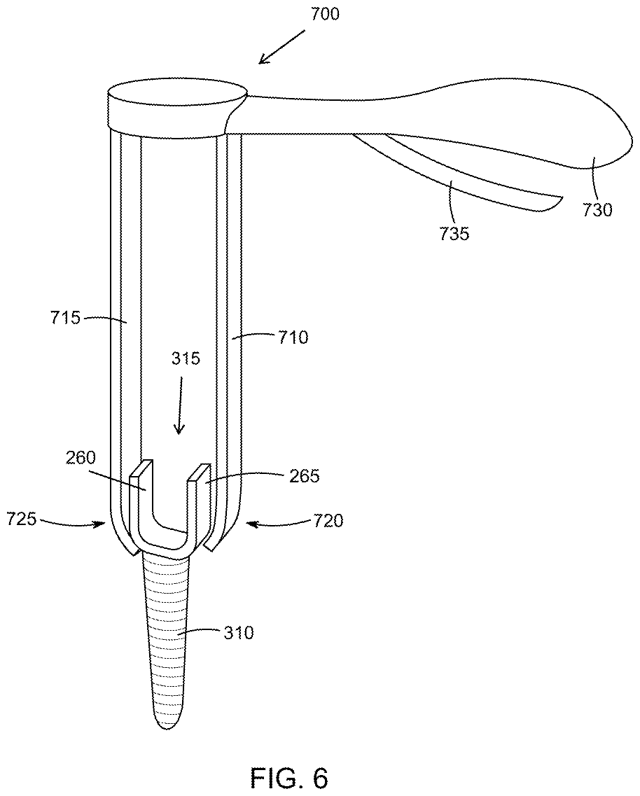

FIG. 6 depicts a screw head gripper securely gripping the screw head of a multi-axial screw to provide an extended placement channel.

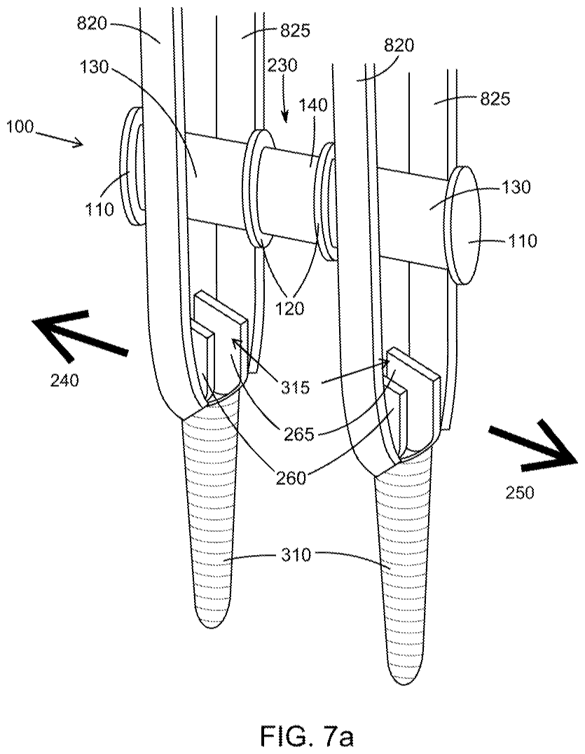

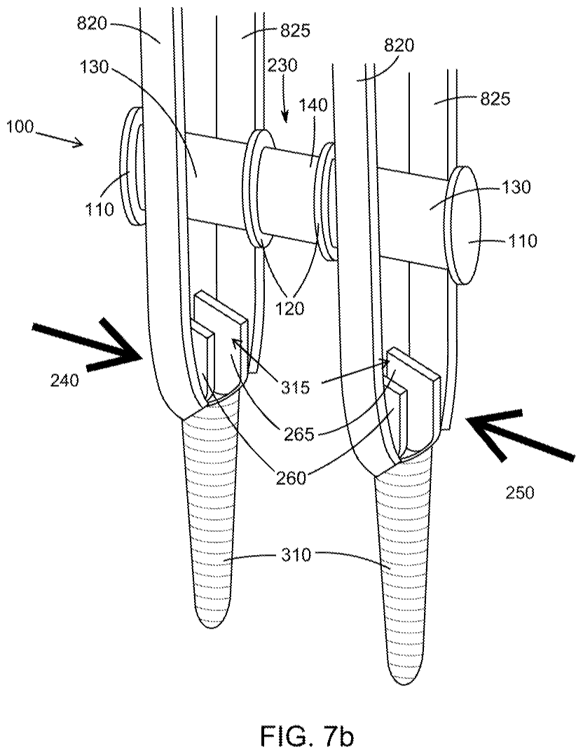

FIG. 7a depicts an embodiment of the capped distraction/compression rod engaging with two screw head grippers that are used to securely grip the screw heads of a set of two multi-axial screws and to provide extended placement channels, and imparting a distraction force.

FIG. 7b depicts an embodiment of the capped distraction/compression rod engaging with two screw head grippers that are used to securely grip the screw heads of a set of two multi-axial screws and to provide extended placement channels, and imparting a compression force.

### DETAILED DESCRIPTION

The embodiments of the present disclosure provide a more efficient system and method for applying compression and distraction forces as part of spinal procedures, particularly involving minimally invasive approaches. Embodiments of the present disclosure include an improved rod that is designed and sized to provide compression/distraction forces during placement and in conjunction with screws implanted into the pedicles of a patient's spine.

Embodiments of the improved rod can include a number of inner segments for engaging with the mounted screw heads. The various inner segments are sized to securely engage with the dimensions of the screw heads. The spacing of the various inner segments can be arranged in a number of combinations to provide compression and/or distraction forces. Preferably, the particular combinations of spacings for any rod is predefined and a range of variations is provided through a set of rods having varying lengths or combinations of spacings at set intervals.

FIG. 1a illustrates the components of a capped distraction/compression rod 100. Capped distraction/compression rod 100 is comprised of 4 components. The rod itself comprises two screw head rod segments 130 of equal length that engage with the screw heads of two screws, and bridging rod segment 140 bridging between the screw heads. Flanking each of the screw head rod segments 130 are an outer end cap 110 and an inner end cap 120.

The outer and inner end caps present a larger cross-sectional profile compared to the screw head rod segment, thus providing limiting walls that define the length of the screw head rod segment and the amount of movement possible when the capped distraction/compression rod is seated within the screw head of a multi-axial screw. Preferably, the dimensions of screw head rod segments 130 are defined to securely engage with the types of screw heads used. Each pair of outer end cap 110 and inner end cap 120 contacts the sides of a respective screw head, restricting or at least limiting the amount of movement possible between the two screws that are engaged by the capped distraction/compression rod 100.

Capped distraction/compression rod 100 preferably has a gentle overall curvature to accommodate the average lumbar lordosis in the spine. Capped distraction/compression rod 100 can be machined from various metals, preferably cobalt chrome.

Bridging rod segment 140 can be of varying lengths, altering the overall length of capped distraction/compression rod 100. Conventionally, the appropriate size for a rod would be measured using a rod template measuring device. In a preferred embodiment, capped distraction/compression rod 100 would provide a defined amount of distraction by providing a length of bridging rod segment 140 that is longer than the distance measured between the screw heads. By “over-sizing” capped distraction/compression rod 100, distraction can be achieved by a distance equal to the difference between the oversized rod and the in-situ measured distance. Alternatively, capped distraction/compression rod 100 would provide a defined amount of compression by providing a length of bridging rod segment 140 that is shorter than the distance measured between the screw heads. By “under-sizing” capped distraction/compression rod 100, compression can be achieved by a distance equal to the difference between the undersized rod and the in-situ measured distance.

Alternatively, inner end caps 120 may be provided by designing bridging rod segment 140 to be a thicker inner section of the capped distraction/compression rod 100, with the thickness varying as needed to securely engage with and seat within the screw head of conventional multi-axial screws. In another embodiment of the present disclosures, the bridging rod segment 140 may comprise a tube-shaped sleeve of a defined thickness and length which can be adjustably positioned on and then fixed to a longer base rod, along with one or more end caps of defined thicknesses and lengths, as parts of the capped distraction/compression rod 100. Alternatively, the bridging rod segment 140 may comprise a thick rod section having a defined length to which a thinner rod with an end cap can be inserted into each end of the bridging rod segment 140 to adjustably position and then affix as parts of the capped distraction/compression rod 100. Those skilled in the arts would recognize additional relative positions, and overall lengths can be customized and achieved.

FIG. 1b illustrates the components of a capped distraction/compression rod 105. The components are identical to the components of capped distraction/compression rod 100 in FIG. 1a, except capped distraction/compression rod 105 incorporates additional numbers of segments 130 and 140 to accommodate an additional screw. The rod itself comprises three screw head rod segments 130 of equal length that engage with the screw heads of three screws, and two bridging rod segments 140 bridging between the screw heads. Flanking the outer two screw head rod segments 130 are an outer end cap 110 and an inner end cap 120. Flanking the inner screw head rod segment 130 are two additional inner end caps 125. Capped distraction/compression rod 105 would be used to span two vertebral levels by connecting three screws.

Similarly with the description above for FIG. 1a, the outer and inner end caps present a larger cross-sectional profile compared to the screw head rod segments, thus providing limiting walls that define the length of the screw head rod segments and the amount of movement possible when the capped distraction/compression rod is seated within the screw head of a multi-axial screw. Preferably, the dimensions of screw head rod segments 130 are defined to securely engage with the type of screw heads used. Each pair of outer end cap 110 and inner end cap 120 contacts the sides of a respective screw head. Similarly, the pair of inner end caps 125 contacts the sides of a respective screw head. Combined, the outer and inner caps restrict or at least limit the amount of movement possible between the three screws that are engaged by the capped distraction/compression rod 105.

In another embodiment of the present disclosures, the bridging rod segments 140 may comprise tube-shaped sleeves of defined thicknesses and lengths which can be adjustably positioned on and then fixed to a longer base rod, along with one or more end caps of defined thicknesses and lengths, as parts of the capped distraction/compression rod 105. Alternatively, the bridging rod segments 140 may comprise thick rod sections having defined lengths to which two thinner rods with end caps can be inserted into one end of each of two of the bridging rod segments 140 to adjustably position and then affix. The other ends of each of the bridging rod segments 140 are then connected to thinner rod inserts that can be adjustably positioned and then affixed as parts of the capped distraction/compression rod 105. Those skilled in the arts would recognize additional numbers of segments, relative positions, and overall lengths can be customized and achieved using combinations of the foregoing parts.

Capped distraction/compression rod 105 also preferably has a gentle overall curvature to accommodate the average lumbar lordosis in the spine. Capped distraction/compression rod 105 also can be machined from various metals, preferably cobalt chrome.

Bridging rod segments 140 can be of varying lengths, and a combination of the same or different lengths respectively, altering the overall length of capped distraction/compression rod 105. Conventionally, the appropriate size for a rod would be measured using a rod template measuring device. In a preferred embodiment, capped distraction/compression rod 105 would provide a defined amount of compression or distraction by providing lengths of bridging rod segments 140 that are different than the distance measured between adjacent screw heads. By “over-sizing” a bridging rod segment 140 of capped distraction/compression rod 105, distraction can be achieved by a distance equal to the difference between the oversized rod and the in-situ measured distance. By “under-sizing” a bridging rod segment 140 of capped distraction/compression rod 105, compression can be achieved by a distance equal to the difference between the undersized rod and the in-situ measured distance.

In one embodiment, both bridging rod segments 140 can be over-sized the same amount to provide equal distraction between the three screws. In another embodiment, bridging rod segments 140 can be over-sized by differing amounts to provide different amounts of distraction between the three screws. In another embodiment, only one of the bridging rod segments 140 can be over-sized providing distraction between one pair of the three screws. The other bridging rod segment 140 can be under-sized to provide compression between the other pair of screws, or it can be sized to provide no compression or distraction force.

In one embodiment, both bridging rod segments 140 can be under-sized the same amount to provide equal compression between the three screws. In another embodiment, bridging rod segments 140 can be under-sized by differing amounts to provide different amounts of compression between the three screws. In another embodiment, only one of the bridging rod segments 140 can be under-sized providing compression between one pair of the three screws. The other bridging rod segment 140 can be over-sized to provide distraction between the other pair of screws, or it can be sized to provide no compression or distraction force.

Alternatively, inner end caps 120 and 125 may be provided by designing bridging rod segments 140 to be thicker inner sections of the capped distraction/compression rod 105, with the thickness varying as needed to securely engage with and seat within the screw head of conventional multi-axial screws.

FIG. 2a illustrates the insertion and integration of capped distraction/compression rod 100 into the tapered portion 270 and 275 of the screw tab extenders 260 and 265. Screw tab extenders 260 and 265 are connected to the screws mounted to the pedicles of a patient's spine. Once the capped distraction/compression rod 100 is inserted appropriately into tapered portions 270 and 275, outer end cap 110 and inner end cap 120 will flank either side of screw tab extenders 260 and 265, allowing bridging rod segment 140 to seat between the pairs of screw tab extenders 260 and 265. Once seated, capped distraction/compression rod 100 will be applied towards the spine and the mounted screw heads in the indicated direction 230. With gentle force inserting an oversized capped distraction/compression rod 100 towards the screw heads, this will provide an incremental distraction force, shown by opposing arrows 240 and 250, ultimately resulting in distraction of the screws, which may be desired for a variety of anatomical or patient-specific indications. Similar insertion and integration can also be accomplished for a capped distraction/compression rod having more than one bridging rod segment, such as shown in FIG. 1b.

FIG. 2b, similarly to the description of FIG. 2a, illustrates the insertion and integration of capped distraction/compression rod 100. In this case, with gentle force inserting an undersized capped distraction/compression rod 100 towards the screw heads, this will provide an incremental compression force, shown by opposing arrows 245 and 255, ultimately resulting in distraction of the screws, which may be desired for a variety of anatomical or patient-specific indications. Similar insertion and integration can also be accomplished for a capped distraction/compression rod having more than one bridging rod segment, such as shown in FIG. 1b.

FIG. 3a illustrates seating and final placement of capped distraction/compression rod 100 across two screw heads of multi-axial screws 310, achieving distraction in the directions shown by arrows 240 and 250. Multi-axial screws 310 have been previously placed in the spinal vertebrae 320. Inner endcaps 120 engage with the screw heads of multi-axial screws 310 and the sizing of the bridging rod segment between endcaps 120 can be varied to provide different amounts of distraction, as described above.

FIG. 3b illustrates seating and final placement of capped distraction/compression rod 100 across two screw heads of multi-axial screws 310, achieving compression in the directions shown by arrows 245 and 255. Multi-axial screws 310 have been previously placed in the spinal vertebrae 320. Inner endcaps 120 engage with the screw heads of multi-axial screws 310 and the sizing of the bridging rod segment between endcaps 120 can be varied to provide different amounts of compression, as described above.

Similarly, FIG. 4a illustrates seating and final placement of capped distraction/compression rod 105 across three screw heads of multi-axial screws 310, achieving distraction in the directions shown by arrows 240 and 250. Multi-axial screws 310 have been previously placed in the spinal vertebrae 320. Inner endcaps 120 and 125 engage with the screw heads of multi-axial screws 310 and the sizing of the two bridging rod segments 410 and 415 can be varied to provide the same or different amounts of distraction, as described above. Alternatively, a combination of distraction and compression, or distraction and no force, can be achieved by different combinations of the respective sizes of the bridging rod segments 410 and 415.

FIG. 4b illustrates seating and final placement of capped distraction/compression rod 105 across three screw heads of multi-axial screws 310, achieving compression in the directions shown by arrows 245 and 255. Multi-axial screws 310 have been previously placed in the spinal vertebrae 320. Inner endcaps 120 and 125 engage with the screw heads of multi-axial screws 310 and the sizing of the two bridging rod segments 410 and 415 can be varied to provide the same or different amounts of compression, as described above. Alternatively, a combination of distraction and compression, or distraction and no force, can be achieved by different combinations of the respective sizes of the bridging rod segments 410 and 415.

FIG. 5a shows a side profile of the end of a capped distraction/compression rod that is seated into the screw head 315 of a multi-axial screw 310. The screw head 315 comprises a U-shaped bracket having two extensions 260 and 265. End caps 110 and 120 flank either side of screw head 315 and the screw head rod segment seats between extensions 260 and 265. Similarly, for a capped distraction/compression rod with more than one bridging rod segment, any inner screw head rod segment (such as one depicted in FIG. 1b between inner end caps 125) would also seat between extensions 260 and 265 on the screw head 315 of a respective multi-axial screw 310.

FIG. 5b shows a top profile of the end of a capped distraction/compression rod that is seated into the screw head 315 of a 310. The screw head 315 comprises a U-shaped bracket having two extensions 260 and 265. End caps 110 and 120 flank either side of screw head 315 and the screw head rod segment 130 seats between extensions 260 and 265. Similarly, for a capped distraction/compression rod with more than one bridging rod segment, any inner screw head rod segment (such as one depicted in FIG. 1b between inner end caps 125) would also seat between extensions 260 and 265 on the screw head 315 of a respective multi-axial screw 310.

FIG. 6 illustrates an alternative embodiment in which screw head extenders are not used. Screw head gripper 700 is a typical device used to securely grip the screw heads 315 of multi-axial screws 310 previously placed in the spinal vertebrae. The gripper extenders 710 and 715 may be tensioned to the screw head using the trigger mechanism 735. In an alternative embodiment of the present disclosure, the gripper extenders 710 and 715 may preferably be designed to accommodate the outer and inner end caps of a capped distraction/compression rod to allow for placement of a capped distraction/compression rod that is compatible with any manufacturer's screw system which already accepts nominal-sized rods.

FIG. 7a illustrates placement of a capped distraction/compression rod 100 using a pair of screw head grippers 700. Once inserted appropriately into the tapered ends of gripper extenders 710 and 715, each pair of outer end cap 110 and inner end cap 120 will flank either side of the extenders for each screw head gripper 700, allowing the bridging rod segment 140 to seat within gripper extenders 710 and 715. Once seated, the capped distraction/compression rod 100 will be applied towards the spine and screw heads 315 in the indicated direction 230. With gentle force inserting an oversized 100 towards the screw heads, this will provide an incremental distraction force, shown by opposing arrows 240 and 250, ultimately resulting in distraction of the screws 310, which may be desired for a variety of anatomical or patient-specific indications.

Similarly, FIG. 7b illustrates placement of a capped distraction/compression rod 100 using a pair of screw head grippers 700. With gentle force inserting an undersized 100 towards the screw heads, this will provide an incremental compression force, shown by opposing arrows 245 and 255, ultimately resulting in compression of the screws 310, which may be desired for a variety of anatomical or patient-specific indications.

Additionally, in an alternative embodiment, more than two screw head grippers may be used. For example, three screw head grippers 700 may be used to place a capped distraction/compression rod having more than one bridging rod segment as depicted in FIG. 1b. As described above, the sizing of the plurality of bridging rod segments can be varied to provide different or the same amounts of distraction/compression. Alternatively, a combination of distraction/compression/no force can be achieved by different combinations of the respective sizes of the bridging rod segments.

Various improvements described herein may be used in conjunction or separately. Although the invention has been described with reference to exemplary embodiments, it is not limited described above. Those skilled in the art will appreciate that numerous changes and modifications may be made to the preferred embodiments above that do not depart from the scope and spirit of the invention, for example, providing distraction or compression between screws placed in the pedicles of the spine or in other parts of the vertebrae. Additionally, the number, size, and combinations of components described herein can be varied and modified without departing from the scope and spirit of the invention.

What is claimed:

1. An improved rod system for applying spinal distraction or compression comprising:
a rod,
a plurality of end caps positioned along a length of the rod
the rod comprising a plurality of spaced segments, each having a predetermined length defined by the positions of the plurality of end caps,
wherein the predetermined length of two or more of the spaced segments substantially corresponds to the size of a slotted screw head of a plurality of screws placed into a plurality of spinal bones so as to substantially secure the rod segment to the corresponding slotted screw head, and
wherein the predetermined length of each of the one or more remaining spaced segments substantially corresponds to a respective target treatment distance between a pair of consecutive screws in the plurality of spinal bones.

2. The improved rod system of claim 1, wherein the predetermined length of at least one of the remaining spaced segments provides a distraction force between the two corresponding consecutive screws.

3. The improved rod system of claim 2, wherein a second predetermined length of a second of the remaining spaced segments provides a second distraction force of different strength between a second pair of corresponding consecutive screws.

4. The improved rod system of claim 1, wherein the predetermined length of at least one of the remaining spaced segments provides a compression force between the two corresponding consecutive screws.

5. The improved rod system of claim 4, wherein a second predetermined length of a second of the remaining spaced segments provides a second compression force of different strength between a second pair of corresponding consecutive screws.

6. The improved rod system of claim 1, wherein the predetermined length of at least a first of the remaining spaced segments provides a distraction force between a first pair of corresponding consecutive screws and the predetermined length of at least a second of the remaining spaced segments provides a compression force between a second pair of corresponding consecutive screws.

7. The improved rod system of claim 1 further comprising a plurality of screw tab extenders, wherein each screw tab extender temporarily connects with the slotted screw head of the corresponding one of the plurality of screws in the spinal bones to provide an extended placement channel leading to the slotted screw head.

8. The improved rod system of claim 1, wherein the rod further comprises a curvature.

9. The improved rod system of claim 1, wherein at least one of the one or more remaining spaced segments resides between two inner end caps positioned to define the corresponding predetermined length.

10. The improved rod system of claim 1, wherein an inner end cap of a predetermined length defines the location and length of one of the one or more remaining spaced segments.

11. An improved rod system for applying spinal distraction or compression comprising:
a base rod,
two outer end caps positioned at the ends of the base rod,
one or more tube-shaped sleeves, each having a predetermined thickness and length and adjustably positionable along the base rod prior to fixation,
wherein each of the one or more tube-shaped sleeves is affixed to the base rod to define a plurality of spaced segments along the length of the base rod, each having a predetermined length defined by the space between consecutive pairs of one of the sleeves with another sleeve or with an end cap,
wherein the predetermined length of two or more of the spaced segments substantially corresponds to the size of a slotted screw head of a plurality of screws placed into a plurality of spinal bones so as to substantially secure the rod segment to the corresponding slotted screw head, and
wherein the predetermined length between a consecutive pair of the two or more spaced segments corresponds to a respective target treatment distance between the corresponding pair of consecutive screws in the plurality of spinal bones.

12. The improved rod system of claim 11, wherein the target treatment distance between one pair of consecutive screws provides a distraction force.

13. The improved rod system of claim 12, wherein the target treatment distance between a second pair of consecutive screws provides a second distraction force of different strength.

14. The improved rod system of claim 11, wherein the target treatment distance between one pair of consecutive screws provides a compression force.

15. The improved rod system of claim 12, wherein the target treatment distance between a second pair of consecutive screws provides a second compression force of different strength.

16. The improved rod system of claim 11, wherein the target treatment distance between one pair of consecutive screws provides a distraction force and the target treatment distance between a second pair of consecutive screws provides a compression force.

17. An improved rod system for applying spinal distraction or compression comprising:
one or more tube-shaped sleeves, each having a predetermined thickness and length,
two outer end caps, each comprising a corresponding rod segment that is insertable and affixable to one end of the one or more tube-shaped sleeves,
wherein each inserted end cap defines a spaced segment between the end cap and the end of the tube-shaped sleeve, the spaced segment having a length that substantially corresponds to the size of a slotted screw head of a plurality of screws placed into a plurality of spinal bones so as to substantially secure the rod segment to the corresponding slotted screw head, and
wherein the predetermined length of each of the tube-shaped sleeve contributes to defining one or more target treatment distances between a corresponding pair of consecutive screws in the plurality of spinal bones.

18. The improved rod system of claim 17 further comprising a connecting rod insertable between the ends of two tube-shaped sleeves, wherein the connecting rod defines a spaced segment between the ends of the two tube-shaped sleeves, the spaced segment having a length that substantially corresponds to the size of a slotted screw head of a plurality of screws placed into a plurality of spinal bones so as to substantially secure the rod segment to the corresponding slotted screw head.

19. The improved rod system of claim 17, wherein the target treatment distance between one pair of consecutive screws provides a distraction force.

20. The improved rod system of claim 17, wherein the target treatment distance between one pair of consecutive screws provides a compression force.
